(19) United States
(12) Patent Application Publication
(10) Pub. No.: US 2016/0338868 A1
(43) Pub. Date: Nov. 24, 2016

(54) Splints for Ankle Dorsiflexion or Plantar Flexion

(71) Applicant: Schultz Grimes, Inc., Yorba Linda, CA (US)

(72) Inventor: Kevin Grimes, Yorba Linda, CA (US)

(21) Appl. No.: 15/158,535

(22) Filed: May 18, 2016

Related U.S. Application Data
(60) Provisional application No. 62/163,157, filed on May 18, 2015.
(60) Provisional application No. 62/163,832, filed on May 19, 2015.

(51) Int. Cl. A61F 5/042; A61F 5/30; A41B 11/00; A61F 5/01

(52) U.S. Cl. CPC A61F 5/042; A61F 5/0111; A61F 5/30; A41B 11/007

(57) ABSTRACT

Devices and methods for stretching and maintaining a configuration of a wearer's feet are provided herein. A splint can comprise a sole support and a sole support cover, and can lack any straps extending between a wearer's ankle or leg and the wearer's foot, or rigid pieces other than the sole support. In some preferred embodiments, the sole support is entirely positionable beneath or at a lower position than the wearer's foot when standing.

This application claims priority to U.S. provisional application No. 62/163,157, filed May 18, 2015, and U.S. provisional application No. 62/163,832, filed May 19, 2015. These and all other extrinsic materials identified herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is splints for supporting a wearer's foot in a flexed configuration.

### BACKGROUND

The background description includes information that can be useful in understanding the present invention. It is not an admission that any of the information provided herein is prior art or relevant to the presently claimed invention, or that any publication specifically or implicitly referenced is prior art.

Night splints can be used to relieve foot pain and treat conditions like plantar fasciitis and tendonitis. Night splints typically work by extending the foot with the toes pointed upwards (dorsiflexion), which stretches the plantar fascia, and other muscles of the foot and leg.

Night splints can be worn during sleep to apply a gentle and constant pressure to the muscles of the foot. However, for many users, night splints are bulky and uncomfortable and can interfere with normal sleep. For example, U.S. Pat. No. 8,409,123 to Hoffmeier et al. provides a night splint that stretches the foot through a stiff boot and an adjustable strap attached near a toe portion and an ankle portion of the boot. Although the '123 patent's splint may effectively stretch the foot, the boot and its numerous components are relatively bulky, which can make it difficult or uncomfortable for the wearer to sleep while wearing the splint.

Another example of known night splints includes U.S. Pat. No. 8,007,456B2 to Stano. Stano's night splint is not as bulky as some other known night splints, but still requires a stiff boot and an ankle portion, which can be uncomfortable, and can interfere with sleep.

Thus, there is still a need for a less bulky and more comfortable splint for supporting a wearer's foot in a flexed configuration.

### SUMMARY

The inventive subject matter provides apparatus, systems, and methods for splint systems that can provide passive stretching, and be worn during sleep to prevent or relieve pain associated with plantar fasciitis. Contemplated splints can advantageously provide greater comfort and mobility because no straps or rigid structure other than a sole support is required. Viewed from another perspective, the splint could comprise only a sole support positionable beneath a wearer's foot, and a sole support cover (e.g., sock, a sheath, straps, a sandal, a shoe, or any other suitable component) that wraps around a sole support and a foot of the wearer, and can keep the sole support substantially in place.

In some aspects of the inventive subject matter, a splint for stretching a wearer's foot is provided. The splint can comprise a sole support that is sized and dimensioned to be positioned below the wearer's foot, and configured to cause the wearer's foot to adjust or flex to a flexed configuration. A sole support cover can also be provided, and be sized and dimensioned to wrap around at least a portion of the wearer's foot and to allow the wearer to passively maintain the flexed configuration of his or her foot.

Contemplated sole supports can have any commercially suitable size and shape (e.g., curvature for dorsiflexion, curvature for plantar flexion, heel end towards the wearer's heel, heel end away from the wearer's heel, mid-portion shaped for the wearer's arch), and be made of any commercially suitable material(s) (e.g., foam, reformable material, rubber, plastic, nylon, fabric). Additionally, sole supports can optionally include one or more of the following features: perforations for breathability; one or more ridges that are configured to apply a continuous localized pressure to a portion of the wearer's foot (e.g., a fasciitis bar); a curvature that is modifiable (e.g., above specific temperatures).

Contemplated sole support covers include, among other things, socks, wraps and straps. In some preferred aspects, the sole support cover can be worn tightly enough to maintain a flexed configuration of the wearer's foot without causing any discomfort or pain to the wearer. The sole support cover can in some aspects be used to control swelling or edema and provide, for example, a compression of between 5-50 mmHg to the wearer's foot when a splint is worn. Additionally or alternatively, the sole support cover can provide varying or graduated compression along its length or width.

It should be appreciated that a sole support can sometimes be used with different sole support covers based on what a user prefers. For example, it is contemplated that a user could purchase a sole support of the inventive subject matter, and use it in combination with one or more socks or ankle wraps he has available in his home. It should also be appreciated that a sole support cover can generally be used with different sole supports. However, in some preferred embodiments, the sole support comes as a kit with one or more sole support covers.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIGS. 1A-1C illustrate a foot in a relaxed configuration and various flexed configurations.

FIG. 2 illustrates a splint for maintaining a plantar flexion configuration.

FIG. 3 illustrates a splint for maintaining a dorsiflexion configuration.

FIG. 4 illustrates another splint for maintaining a dorsiflexion configuration.

FIG. 5 illustrates yet another splint for maintaining a dorsiflexion configuration.

FIG. 6 illustrates a sole support having a heel stop.

FIGS. 7A-7C illustrate different sole supports having different shapes.

FIG. 8 illustrates a sole support having a fasciitis bar.

FIG. 9 illustrates a sole support having perforations and a pressure providing bump.

FIG. 10 illustrates a sole support having a removable pressure providing bump.

FIGS. 11A-11C illustrate various pressure providing bumps that can be removably attached to the sole support of FIG. 10.

FIGS. 12A-12D illustrate a modifiable sole support.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The inventive subject matter provides apparatus, systems, and methods for splint systems that can provide passive stretching, and be worn during sleep to prevent or relieve pain associated with plantar fasciitis. Contemplated splints can advantageously maintain a flexed configuration of a foot or ankle without any active effort of the user. Flexed configurations can include a dorsiflexion (or toe-up, or stretched fascia) configuration, and a plantar flexion (or toe-down) configuration.

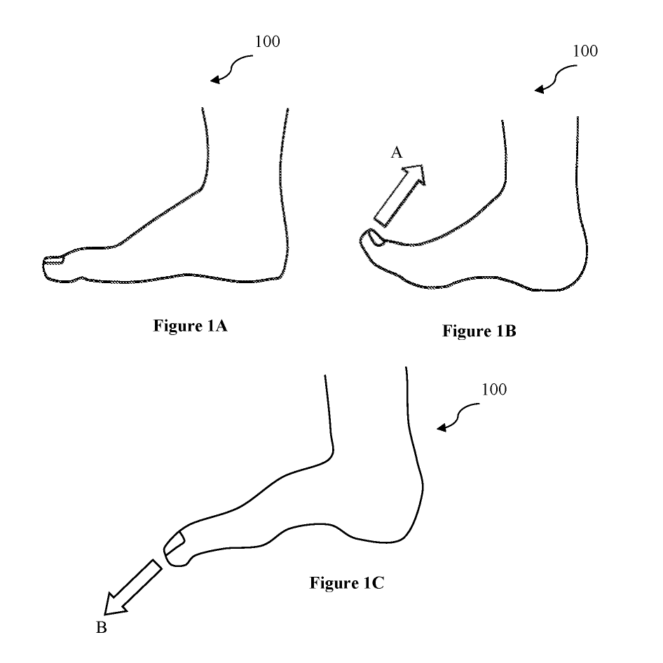

FIG. 1A illustrate a foot 100 in a neutral, resting, non-flexed configuration. FIG. 1B illustrates foot 100 in a dorsiflexion configuration wherein the wearer's toes are flexed towards the wearer's shin bone. FIG. 1C illustrates foot 100 in a plantar flexion configuration wherein the toes are pointed downwards and flexed towards the wearer's sole. It should be appreciated that people have feet that are uniquely shaped (e.g., different arch types, sizes, curvatures, widths), and that the plantar flexion and dorsiflexion range of motion can vary from person to person. Some contemplated splints and sole supports can advantageously maintain a range of different flexed positions, and accommodate a range of sizes and shapes.

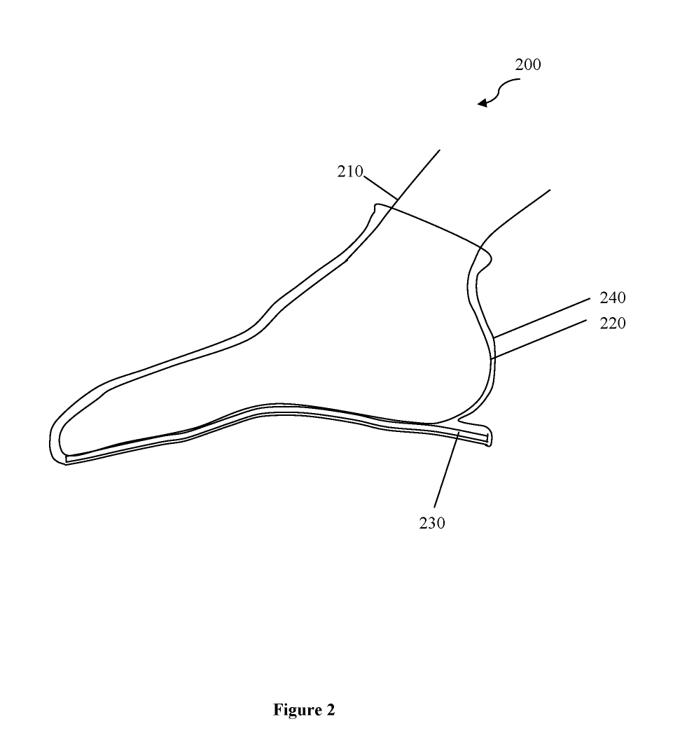

FIG. 2 illustrates a splint 200 that maintains a plantar flexion configuration of a foot. Plantar flexion can occur when the dorsum of the foot lengthens in line with the leg, or points downward. Splint 200 is worn on a wearer's foot 220, and causes ankle 210 to move away from its neutral angle by a range of, for example, between 1-50°. Splint comprises a rigid sole support 230 that is sized and dimensioned to be positioned below foot 220, and a sock of other sole support cover 240 that is sized and dimensioned to wrap around sole support 230 and maintain the flexed configuration of the wearer's foot 220 in a desired plantar flexion configuration.

In the plantar flexion configuration, it is contemplated that at least one of a user's toes, foot, or ankle joint can be modified from its neutral configuration by between 10-90°, between 10-55°, between 15-45°, between 20-40°, between 55-75°, between 60-70°, or any other suitable amount. Viewed from another perspective, the wearer's leg and the wearer's foot can form an angle of between, for example, 90-180°, 90-160°, 145-165°, or 150-160° in the plantar flexion configuration. Viewed from yet another perspective, the front of the wearer's leg and the top of the wearer's foot can form an angle of between, for example, 90-180°, 90-160°, 145-165°, 150-185°, or 150-160° in the plantar flexion configuration.

As illustrated, sole support 230 has a heel end that extends beyond and away from the wearer's foot 220, and is shaped to directly contact at least 90% of the bottom surface of the wearer's foot 220. In some embodiments, even where the sole support extends across the entire bottom surface of the wearer's foot (or across between 80-100% of the bottom surface or sole of the wearer's foot), the sole support could have a different curvature (e.g., S-shaped) and be shaped to contact between 10-100%, between 50-100%, between 80-100%, between 90-100%, between 50-75% or even 25-75% of a bottom surface of the wearer's foot.

Although it is generally preferred that a sole support is rigid or semi-rigid, and can flex without breaking, all suitable material or materials are contemplated, including for example, a polycarbonate, a carbon fiber, fiberglass, ABS, a hard plastic, a rubber, a foam, a plastic, or a metal. Hardness can be measured using any suitable hardness scale including one of the Shore hardness scales. The Shore 00 hardness scale is used to measure soft substances including gels or foams. The Shore A hardness scale is used to measure substances having an intermediate hardness including rubbers. The Shore D hardness scale is used to measure harder substances including plastics. Although the preceding Shore hardness scales are used for different various substances, the scales all overlap at the low end of their spectrums. In some embodiments, sole support 230 can have a Shore D hardness of at least 10, at least 20, at least 30, at least 40, at least 50, at least 60, at least 70, or even 80 or more. Additionally or alternatively, sole support 230 can have a Shore A hardness of at least 30, at least 40, at least 50, or even at least 90 or more. It is contemplated that sole support 230 can be sufficiently rigid such that it does not deform more than 5% when worn by a wearer overnight. This allows sole support 230 to flex the foot so that dorsiflexion or plantar flexion is achieved.

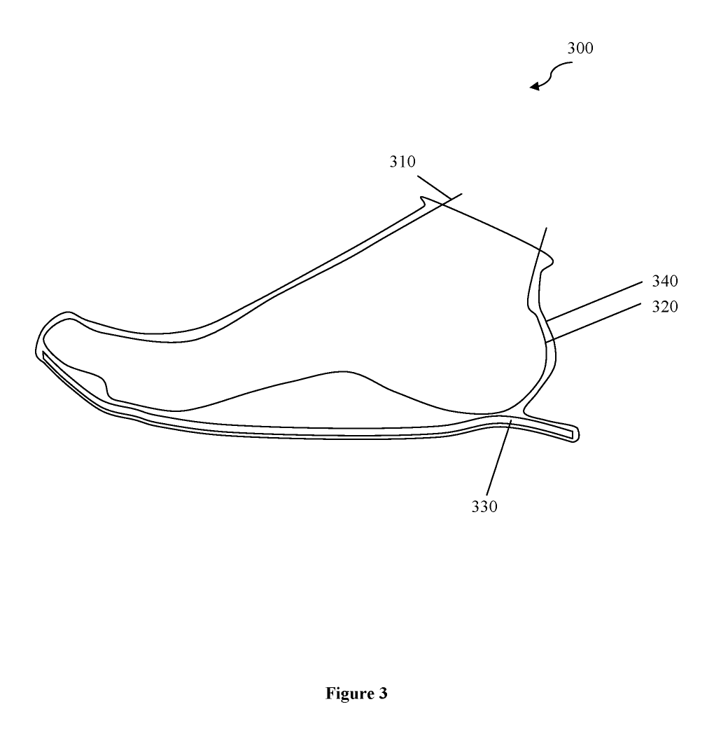

FIG. 3 illustrates a splint 300 that maintains a dorsiflexion configuration of a foot 320. Dorsiflexion can occur when the dorsum of the foot is moved towards the anterior surface of the leg. Splint 300 is worn on a wearer's foot 320, and causes ankle 310 to move away from its neutral angle by a range of, for example, between 1-20°. Splint comprises a rigid sole support 330 that is sized and dimensioned to be positioned below foot 320, and a sock of other sole support cover 340 that is sized and dimensioned to wrap around sole support 330 and maintain the flexed configuration of the wearer's foot 320 in a desired dorsiflexion configuration.

In the dorsiflexion configuration, it is contemplated that at least one of a user's toes, foot, or ankle joint can be modified from its neutral configuration by between 1-25°, between 5-20°, between 5-15°, between 10-15°, or any other suitable amount. Viewed from another perspective, the wearer's leg and the wearer's foot can form an angle of between, for example, 70-85°, 65-90°, or any other suitable angle when in the dorsiflexion configuration.

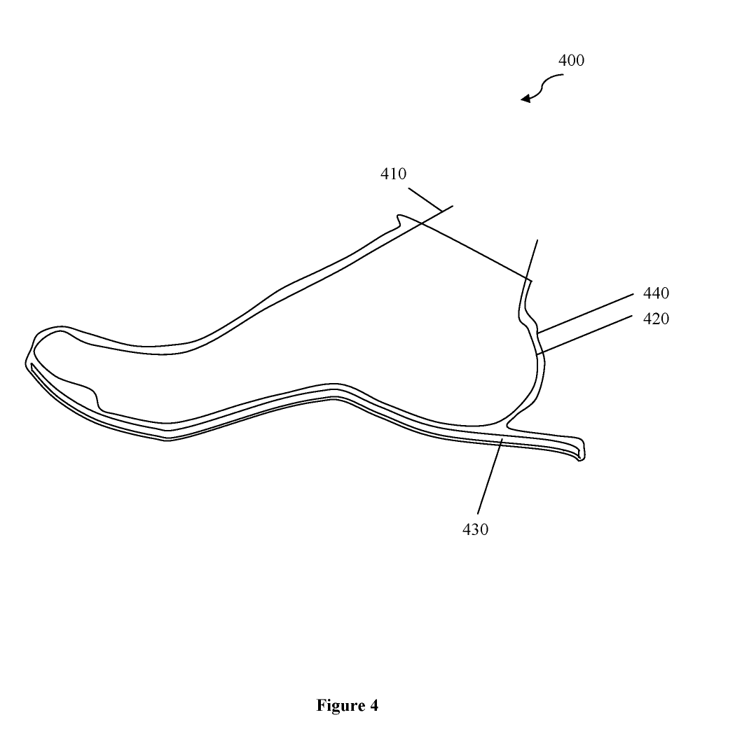

In the embodiment illustrated in FIG. 3, sole support 330 has a modified S-shape, and has a mid-portion that does not directly contact the arch of the wearer's foot 320. However, it is contemplated that in some other embodiments, the sole support can be further curved such that it is configured to directly contact the arch of the wearer (as shown in FIG. 4).

The heel end of sole support 330 is curved away from the wearer's heel, and can act as a “kickstand” that keeps the foot in a desired position when the splint is worn while the user is lying down. Alternatively, the user might want the heel end to angle upwards towards the wearer's heel to, for example, better align with the heel portion of the user's foot (as shown in FIG. 5).

FIG. 4 illustrates yet another splint 400 that causes ankle 410 dorsiflexion, and maintains a dorsiflexion configuration of a foot 420. Splint 400 includes a sole support 430 configured to be positioned under the wearer's foot 420, and a sole support cover 440 that entirely wraps around the sole support and upper foot of the wearer. Unlike sole support 330 of splint 300, sole support 430 has a mid-portion curvature that directly contacts the arch of the wearer's foot. The height of the mid-portion (e.g., when the lower or bottom surface of the sole support is placed against a flat horizontal surface) can be at least 5 mm, at least 10 mm, at least 15 mm, at least 20 mm, at least 25 mm, or even greater.

FIG. 5 illustrates yet another splint 500 that causes ankle 510 dorsiflexion, and maintains a dorsiflexion configuration of a foot 520 via sole support cover 540. Sole support 530 is a modified C-shape, and includes end portions that are slightly curved away from the wearer's foot to prevent pinching.

While the above described embodiments are generally directed to splints having a sock as a sole support cover, it should be appreciated that a wrap, strap, or other object(s) can be used to maintain a position of the sole support relative to the wearer's foot, and assist in maintaining a flexed configuration of the wearer's foot.

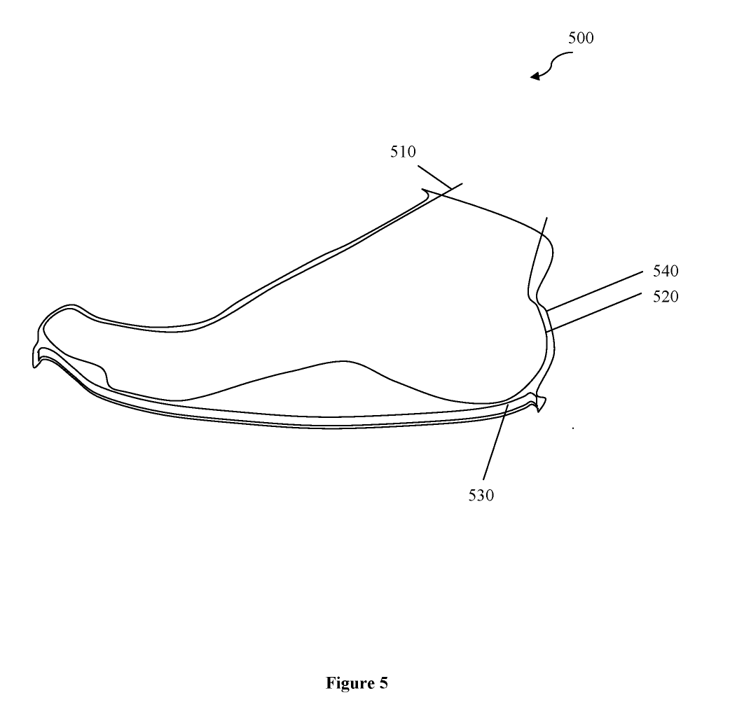

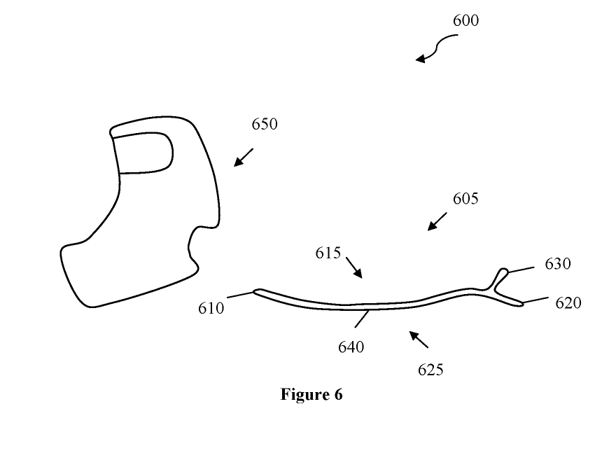

FIG. 6 illustrates such an alternative splint 600. Splint 600 includes a sole support 605 having an upper surface 615, a bottom or lower surface 625, a toe end 610, heel end 620, and a mid-portion 640 that extends between from the toe end to the heel end. Sole support 605 further includes a heel stop 630 that is positioned near heel end 620, and configured to prevent a wearer's foot from sliding back past stop 630. Splint 600 also includes an ankle wrap 650 that is configured to maintain the position of sole support 605 on a wearer's foot, and maintain a dorsiflexion configuration of a wearer's foot when worn with sole support 605. Advantageously, no other components are needed to cause or maintain dorsiflexion, which allows for a wearer's increased mobility and comfort when compared to some known night splints.

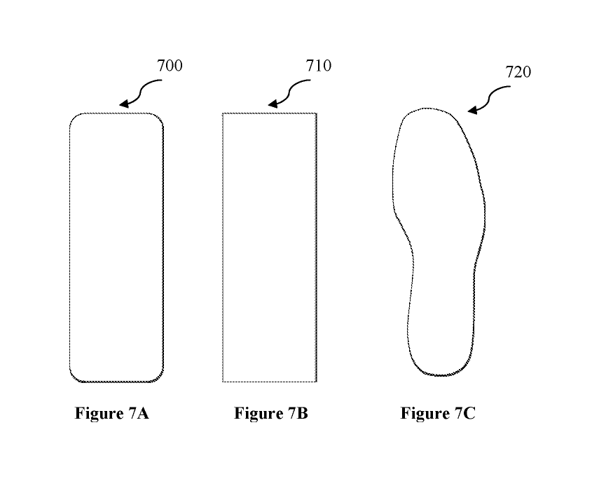

FIGS. 7A-7C illustrate some contemplated shapes for sole supports of the inventive subject matter. FIG. 7A illustrates a top surface of a sole support 700, which is the surface that is configured to contact the bottom of a wearer's foot. Sole support 700 has a modified rectangular shape with rounded edges to prevent a sole support cover from becoming damaged. FIG. 7B illustrates a sole support 710 having a rectangular shape, and FIG. 7C illustrates a sole support 720 having a shape similar to a bottom surface of a wearer's foot.

Some preferred sole supports will be oversized compared to a wearer's foot, have a length greater than the length of the wearer's foot, and have a width greater than the width of the wearer's foot. However, it is also contemplated that the sole support can be smaller, narrower or shorter than the wearer's foot.

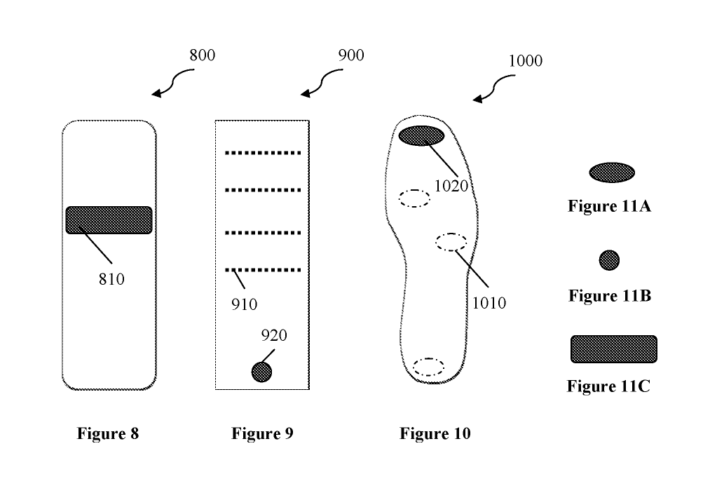

In some embodiments, sole supports can include one or more additional features to provide additional benefits to a wearer. For example, as shown in FIG. 8, sole support 800 includes a fasciitis bar 810 that is configured to apply a continuous pressure to the plantar fascia of a wearer's foot. As another example, as shown in FIG. 9, sole support 900 includes perforations 910 so that air can pass through the sole support 900 to the wearer's foot, and further includes a projection, ridge or bump that provides continuous pressure to a heel of a wearer. As yet another example, FIG. 10 illustrates a sole support 1000 including fasteners (e.g., 1010) to which a padded pressure piece 1020, a padded ridge of FIG. 11A, an acupressure bead/piece of FIG. 11B, or a fasciitis bar of FIG. 11C can be removably coupled.

The projections, bumps, pressure piece, ridges and fasciitis bar can be made of any suitable material, including for example, a foam, a pad, a gel, a plastic, or any other suitable material. Additionally or alternatively, the projections, bumps, pressure piece, ridges and fasciitis bar can have a height of between 1 mm-50 mm, between 1 mm-25 mm, between 5-25 mm, between 10-25 mm, or any other suitable height.

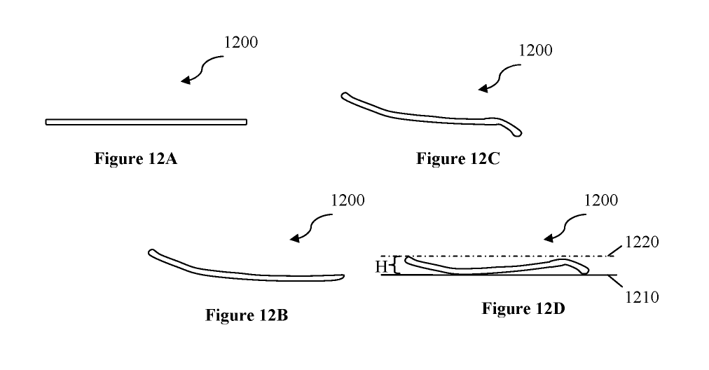

FIGS. 12A-12D illustrate a sole support that is made from a reformable or moldable material (e.g., thermoformable material), and is custom molded to fit a wearer's foot. Sole support 1200 can start as a flat piece of material, as shown in FIG. 12A, having a substantially uniform thickness (i.e., a thickness that does not vary by more than 10% throughout at least 80% of the length and at least 80% of the width of the sole support). The sole support can have any suitable thickness, including for example, between 2-20 mm, between 2-15 mm, between 2-10 mm, or between 5-15 mm. Viewed from another perspective, the entire sole support could have a thickness of between 1/16-½ inch, between 1/16-¼ inch, between ⅛-¼ inch, or any other suitable thickness.

Under the right conditions, the sole support 1200 can be moldable, and the toe end of the sole support can be curved up for dorsiflexion, or down for plantar flexion. The heel end can be curved up to contact the heel of the wearer's foot, or can be curved downwards away from where the wearer's heel would be positioned. If it is desirable to change the configuration of the sole support, it is contemplated that the sole support can be reformed or modified as desired. Contemplated sole supports could be configured to have curvatures and angles to allow for different degrees of dorsiflexion and plantar flexion of the foot.

After sole support 1200 is molded to fit the wearer's foot, and set on a flat horizontal surface 1210, it is contemplated that the height of the sole support (i.e., the distance between the flat surface and the uppermost point of the sole support configured to contact the wearer's foot) can be between 1 cm-15 cm, between 1 cm-10 cm, between 1-8 cm, between 2-8 cm, between 2-6 cm, at least 2 cm, at least 3 cm, at least 4 cm, at least 5 cm, or any other suitable height.

As used herein, the term “splint” should be interpreted broadly to include an object made at least in part of a rigid or semi-rigid material and used to support the foot. In some embodiments, a splint can be used to achieve dorsiflexion or plantar flexion of the ankle.

Thus, various night splints and methods of using night splints have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A splint for stretching a wearer's foot, comprising:
a sole support and a sole support cover;
wherein the sole support is sized and dimensioned to be positioned below the wearer's foot, and configured to cause the wearer's foot to flex to a flexed configuration; and
wherein the sole support cover is sized and dimensioned to wrap around at least a portion of the sole support and the wearer's foot, and to maintain the flexed configuration of the wearer's foot.

2. The splint of claim 1, wherein the sole support has a toe end and a heel end, wherein when the splint is worn:
the heel end is sized and dimensioned to extend beyond the wearer's foot; and
the heel end is curved away from the wearer's foot.

3. The splint of claim 1, wherein the sole support is made of a hard plastic, and wherein the sole support is sized and dimensioned to wrap around the entire sole support and the wearer's foot.

4. The splint of claim 3, wherein the sole support is perforated.

5. The splint of claim 1, wherein the flexed configuration is a toe-down configuration.

6. The splint of claim 5, wherein the sole support has a toe end, a heel end, and a mid-portion extending between the toe end and the heel end, and wherein the mid-portion has a curvature having a height of at least 15 mm.

7. The splint of claim 5, wherein the sole support has an upper surface and a lower surface, and wherein the upper support comprises a first portion and a second portion, and wherein the first portion is at least 15 mm above a second portion when the lower surface is positioned on a flat, horizontal surface.

8. The splint of claim 7, wherein the first portion comprises a portion of the sole support configured to be positioned at an arch of the wearer's foot, and wherein the second portion comprises a heel end.

9. The splint of claim 1, wherein the flexed configuration is a toe-up configuration.

10. The splint of claim 5, wherein when the wearer's foot is in the flexed configuration, a front surface of a leg of the wearer and a top surface of the wearer's foot form an angle of between 140-180 degrees.

11. The splint of claim 1, wherein the sole support cover comprises a sock.

12. The splint of claim 1, wherein the sole support cover provides a pressure of between 10-30 mmHg to the wearer's foot when the splint is worn.

13. The splint of claim 1, wherein the sole support comprises a padding.

14. The splint of claim 1, wherein the sole support comprises a first ridge configured to apply a continuous localized acupressure to a portion of the wearer's foot, and wherein the first ridge has a height of at least 10 mm.

15. The splint of claim 1, wherein the sole support comprises a fasciitis bar having a height of at least 15 mm.

16. The splint of claim 1, wherein a curvature of the sole support can be modified.

17. The splint of claim 1, wherein the sole support has a Shore D durometer hardness of at least 10.

18. The splint of claim 1, wherein the sole support has a heel end that is configured to extend beyond a heel of the wearer's foot when the splint is worn, and wherein the heel end has a curvature that can be modified.

19. The splint of claim 1, wherein the sole support is made of a material that is moldable above a first temperature, and rigid below the first temperature.

20. The splint of claim 1, wherein the sole support has a substantially uniform thickness.
